(19) United States
(12) Patent Application Publication
(10) Pub. No.: US 2013/0253427 A1
(43) Pub. Date: Sep. 26, 2013

(54) Drug Delivery Device

(71) Applicants: Zdenek Cerman, Frankfurt am Main (DE); Andreas Bode, Frankfurt am Main (DE)

(72) Inventors: Zdenek Cerman, Frankfurt am Main (DE); Andreas Bode, Frankfurt am Main (DE)

(73) Assignee: SANOFI-AVENTIS DEUTSCHLAND GMBH, Frankfurt am Main (DE)

(21) Appl. No.: 13/991,561

(22) PCT Filed: Dec. 7, 2011
(86) PCT No.: PCT/EP11/72139

§ 371 Date: Jun. 4, 2013

(30) Foreign Application Priority Data
Dec. 9, 2010 (EP) 10194420.5

(51) Int. Cl. A61M 5/50

(52) U.S. Cl. CPC A61M 5/5086
USPC 604/111

(57) ABSTRACT

A component for a drug delivery device comprises a key validation part and a safety part. The key validation part is configured to interact with a key part of another component of the drug delivery device during the assembly of the drug delivery device. In order to support the use of matching components, the safety part is configured to have a different impact if the key part is matched to the key validation part compared to if the key part is not matched to the key validation part.

### CROSS REFERENCE TO RELATED APPLICATIONS

The present application is a U.S. National Phase Application pursuant to 35 U.S.C. §371 of International Application No. PCT/EP2011/072139 filed Dec. 7, 2011, which claims priority to European Patent Application No. 10194420.5 filed Dec. 9, 2010. The entire disclosure contents of these applications are herewith incorporated by reference into the present application.

### FIELD OF INVENTION

The invention relates to the field of drug delivery devices, and more specifically to the assembly of components of drug delivery devices.

### BACKGROUND

A drug delivery device may comprise for instance a delivery and dosing apparatus and an exchangeable cartridge, the cartridge being optionally arranged in addition in a cartridge holder.

A delivery and dosing apparatus, like an injection pen, can be driven electromechanically by a motor, which drives a transmission to force a liquid substance, for instance a drug or medicament, out of an attached cartridge.

The cartridge may have a cylindrical body made of glass, plastics, and/or the like. A first end of the cartridge may be closed by a septum which may be pierced by a needle. A second end of the cartridge may be closed by a plunger which is movable inside the cylindrical body of the cartridge to force out the liquid substance or medicament through the needle in the pierced septum.

The construction of a delivery and dosing apparatus can be adapted specifically to a particular substance that is to be delivered. For example, some delivery and dosing apparatus may be adapted to supply a higher force for use with a liquid substance having a higher viscosity, while another delivery and dosing apparatus may be adapted to supply a smaller force for use with a liquid substance having a smaller viscosity, to deliver the same dosage of substance. Different forces can be achieved by different designs of a motor moving the plunger, or by using plungers of different materials, which results in different amounts of friction.

Furthermore, a user may use different drug delivery devices, for instance differently colored drug delivery devices, for applying medicaments having a fast or slow efficacy, respectively.

### SUMMARY

For these and other reasons, it may be important to ensure that a specific drug delivery device is used with a particular cartridge only.

A component for a drug delivery device is presented. The component comprises a key validation part configured to interact with a key part of another component of the drug delivery device during an assembly of the component with the other component. The component further comprises a safety part configured to have a different impact if the key part is matched to the key validation part compared to if the key part is not matched to the key validation part.

Moreover, a drug delivery device is presented, which comprises such a component and such another component.

The use of a key system for a drug delivery device has the advantage that it enables a large variety of codings. Providing a safety part in combination with a key system has the advantage that the use of matching components can be facilitated.

Exemplary embodiments of the invention are defined in the appended claims, as well as in the following description.

In an exemplary embodiment, the component is a drug delivery apparatus, a drug dosing and delivery apparatus, a cartridge configured to comprise a drug, a cartridge holder, or any combination of two of such elements. For instance, a key validation part could belong to a drug delivery apparatus or a drug dosing and delivery apparatus while the key part could belong to a cartridge holder, or vice versa. Furthermore, a key validation part could belong to a drug delivery apparatus or a drug dosing and delivery apparatus while the key part could belong to a cartridge, or vice versa. Furthermore, a key validation part could belong to a cartridge holder while the key part could belong to a cartridge, or vice versa. It could also be provided that three or more elements of a drug delivery device have to be matched to each other to obtain a functional device. This could be achieved by using separate key systems between respective two of the elements, or by splitting up key validation part and/or safety part and/or key part to two of the elements, and/or by adding the safety part to another element than the key validation part, or the like. The drug delivery apparatus or the drug dosing and delivery apparatus may be an injection pen, an infusion pump, an inhaler and/or the like.

The key system could be distributed for example, but not exclusively, to cartridge and dosing/delivery apparatus, to cartridge holder and dosing/delivery apparatus, or to cartridge and cartridge holder. Its use is thus very flexible.

A key validation part may comprise for instance one or more pins attached to a respective biasing element. A key part could then be allowed to move each pin against the biasing element force by a distance that depends on the shape of the key part. Only a matched key part would move the pin or pins by a predetermined distance to a release position. The number of possible combinations for dedication increases significantly with the number of employed pins as well as with the range of possible predetermined distances, similarly as in the case of a pin tumbler lock. Such a biasing element could comprise for instance a spring and/or an elastic plastic arm, or the like.

Alternatively, a key validation part may comprise for instance at least one proximity sensor. Each proximity sensor could be configured to detect whether a key part comes to lie within or at a predetermined distance to the proximity sensor during an assembly of the component with the other component. A hardware circuit or a software-based processor could then determine whether the key part of the other component matches the key validation part by evaluating the output of the at least one proximity sensor. The number of possible combinations for dedication increases significantly with the number of employed proximity sensors as well as with the range of possible predetermined distances.

The safety part could be implemented in various ways. In an exemplary embodiment, it could comprise a blocking element that enables, as the different impact, a full assembly of the device only if key part and key validation part match. The blocking element could interact directly and mechanically with the key validation part. In this case, a key validation part interacting with a matching key part could move the blocking element from a blocking to a non-blocking position or allow the blocking element to move from a blocking to a non-blocking position. Only in case of such a non-blocking position, a full assembly of the components is possible. Alternatively, the state of a key validation part could be monitored electronically and the blocking element could be controlled electronically.

In another exemplary embodiment, the safety part could comprise a blocking element that enables, as the different impact, the operation of the drug delivery device only if key part and key validation part match. Enabling the operation could comprise for instance energizing the device or otherwise setting it into a ready to start state. The operation could be enabled electronically, electromagnetically, electromechanically and/or mechanically.

In another exemplary embodiment, the safety part could further comprise an indicator indicating to a user, as the different impact, that the other component is a matching component, or it could comprise an indicator indicating, as the different impact, the type of one of at least two possible matching other components. The indicator could provide in particular a visual indication, using for instance one or more light emitting diodes, a display or simply an arrow to any of one or more markings. But it could equally provide any other kind of indication, like a haptic indication or an audible indication.

The key part of the other component could be covered by a cap as long as the other component is not in use for protecting the key part as well as for informing a user about the contained medicament.

Exemplary embodiments of the invention will be described in more detail in the following with reference to drawings.

### BRIEF DESCRIPTION OF THE DRAWINGS

FIG. 1 is a schematic diagram presenting matching components of a drug delivery device according to a first embodiment;

FIG. 2 is a schematic diagram presenting the matching components of FIG. 1 in a first interaction state;

FIG. 3 is a schematic diagram presenting the matching components of FIG. 1 in a second interaction state;

FIG. 4 is a schematic diagram presenting non-matching components of a drug delivery device according to the first embodiment in a blocking state;

FIG. 5 is a schematic diagram presenting components of a drug delivery device according to a second embodiment;

FIG. 6 is a schematic diagram presenting a key validation part of the drug delivery device of FIG. 5;

FIG. 7 is a schematic diagram presenting the key validation part of FIG. 6 when interacting with a matched key part;

FIG. 8 is a schematic diagram presenting components of a drug delivery device according to a third embodiment;

FIG. 9 is a schematic diagram presenting details of the drug delivery device of FIG. 8;

FIG. 10 is a schematic diagram presenting a variation of the drug delivery device of FIGS. 8 and 9 including an indicator;

FIG. 11 is a schematic diagram presenting components of a drug delivery device according to a fourth embodiment; and

FIG. 12 is a schematic diagram presenting components of a drug delivery device according to a fifth embodiment.

### DETAILED DESCRIPTION

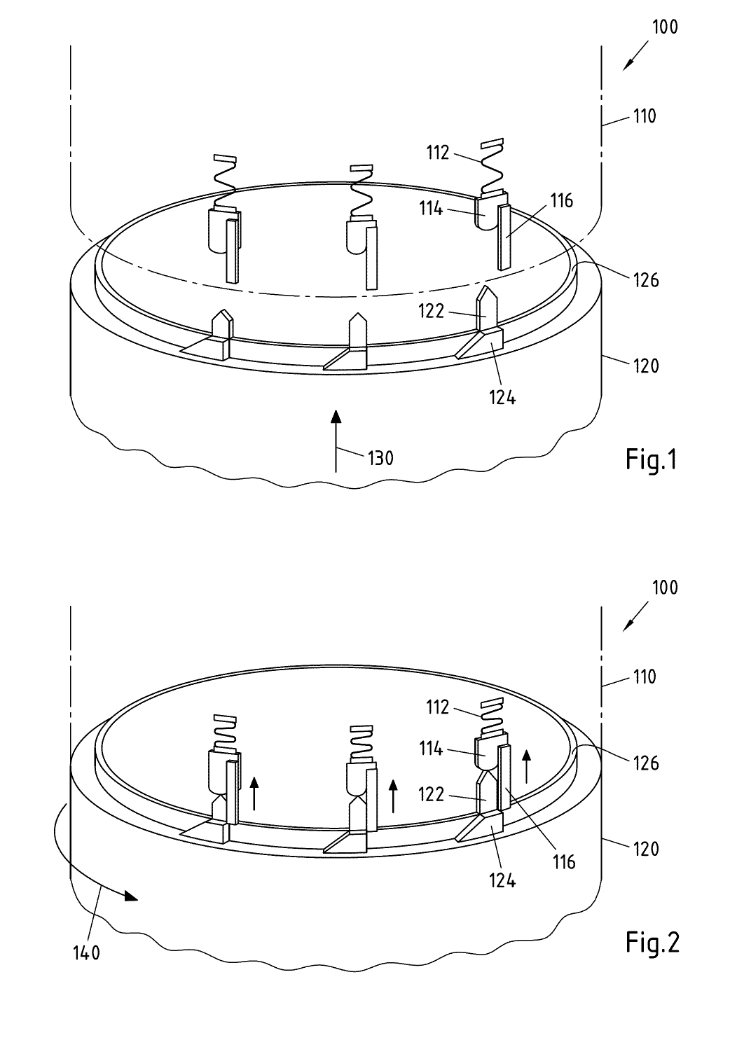

FIGS. 1 to 4 illustrate an assembly of a drug delivery device 100 in accordance with an exemplary first embodiment of the invention.

FIG. 1 is a schematic diagram, which presents selected components of the drug delivery device 100. The drug delivery device 100 could be for instance an injection pen, an infusion pump, an inhaler, and/or the like. In an example embodiment, the drug delivery device 100 may be an insulin pen or an injection pen for another drug or medicament.

The presented components comprise a dosing and delivery apparatus 110 depicted in the upper part of the diagram and a cartridge holder 120 depicted in the lower part of the diagram. The dosing and delivery apparatus 110 and the cartridge holder 120 including a cartridge have to be assembled to obtain a functional drug delivery device 100. The components can only be assembled, in case a key part of the cartridge holder 120 is matched to a key validation part of the delivery apparatus 110.

The dosing and delivery apparatus 110 is shown to have a transparent housing for illustration purposes only. It comprises conventional components of a dosing and delivery apparatus not shown, for example a motor for driving a plunger to force a drug out of a cartridge and a battery for powering the motor, etc.

The dosing and delivery apparatus 110 comprises in addition at its lower end, facing the cartridge holder 120, a key validation part. The key validation part includes three springs 112 that are fixed at an upper end to the side wall of the dosing apparatus. The key validation part includes in addition three pins 114, each being attached to the lower ends of one of the three springs 112. The dosing apparatus 110 comprises in addition a safety part. The safety part comprises three small rods 116, each rod being fixed to a respective one of the pins 114 at the side facing the housing of the dosing apparatus 110 such that each rod 116 extends downwards towards the cartridge holder 120 on the right hand side of and slightly closer to the housing of the dosing apparatus 110 than a respective pin 114.

The cartridge holder 120 comprises a cartridge with a particular drug, like a particular insulin product. The cartridge holder 120 further comprises at the upper end facing the dosing apparatus 110 key elements 122 and return elements 124. The key elements 122 form a key part of the cartridge holder 120. They comprise by way of example three pins extending in direction of the dosing apparatus 110. The height of the key elements 122 is specific to a particular type of drug in the cartridge. They may be arranged on top of a ring-structure 126 at the upper end of the cartridge holder 120 that is flush with the inner wall of the cartridge holder 120 but recessed to the outer wall of the cartridge holder 120. The key elements 122 have the same radial distance to a center of the cartridge holder 120 as the pins 114, when cartridge holder 120 and dosing and delivering apparatus 110 are aligned. The return elements 124 have the form of a right-angled trapezoid with the short one of the parallel sides facing upwards and the slope running downwards to the left. They are arranged on top of the outer wall of the cartridge holder 120 and have the same height as ring-structure 126. The upper, short parallel side of the return elements 124 is moreover horizontally aligned with the bottom of a respective one of the key element 122. The return elements 124 have the same radial distance to a center of the cartridge holder 120 as the rods 116, when cartridge holder 120 and dosing and delivering apparatus 110 are aligned.

As indicated by arrow 130, the upper part of the cartridge holder 120 first has to be approached to the lower part of the dosing delivery apparatus 110. Some complementary alignment elements at dosing and delivery apparatus 110 and cartridge holder 120, not shown, are provided for ensuring alignment.

FIG. 2 is a schematic diagram, which presents the same selected components of the drug delivery device 100 as FIG. 1. Same elements are denoted with same reference signs.

In this assembly stage, the upper part of the cartridge holder 120 has been approached as far as possible to the lower part of the dosing and delivery apparatus 110. As can be seen, the pins 114 of the dosing and delivery apparatus 110 are in contact with the key elements 122 of the cartridge holder 120, and the pins 114 have been pushed upwards compressing the springs 112. Since the rods 116 are connected to the pins 114, they have been moved upwards by the same amount as the pins 114.

Since the key elements 122 are matched to the pins 114 of the dosing apparatus 110, the bottom of all rods 116 stays above the return elements 124 of the cartridge holder 120.

As indicated by arrow 140, the cartridge holder 120 now has to be rotated in a counter clockwise rotation, in order to complete the assembly of the drug delivery device 100.

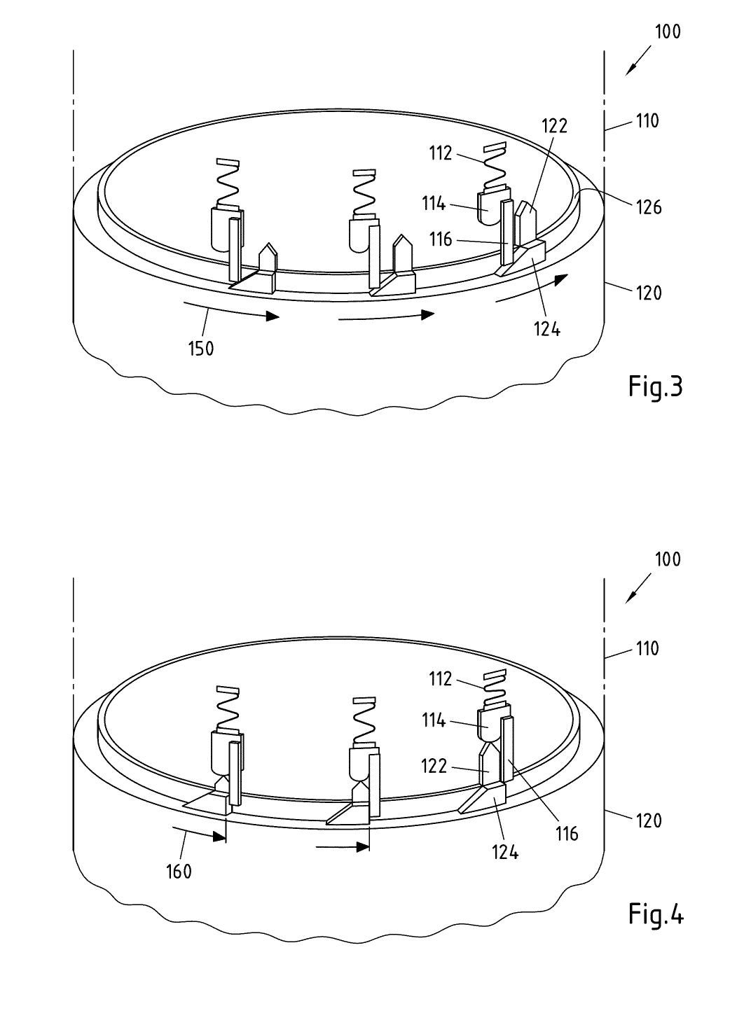

FIG. 3 is a schematic diagram, which presents again the same selected components of the drug delivery device 100 as FIG. 1. Same elements are denoted with same reference signs. In the depicted assembly stage, the return features 124 of the cartridge holder 10 are being rotated past the rods 116 of the dosing apparatus 110, as indicated by arrows 150. In this process, the cartridge holder 120 is screwed in to the dosing apparatus 110, or otherwise connected. Any kind of standard assembly process or connections could be used to this end, including threads, luer lock, etc.

As soon as the rods 116 get into the region of the slope of the return features 114, the springs 112 begin to expand again, as shown in FIG. 3. The slope of the return features 114 ensures that a reverse rotation is enabled for disconnecting the components 110, 120 again.

FIG. 4 is a schematic diagram, which presents again similar selected components of a drug delivery device 100 as FIG. 1. Corresponding elements are denoted with same reference signs. In this case, however, the key elements 122 of the cartridge holder 120 are not matched to the key validation part 112, 114 of the dosing and delivery apparatus 110.

When approaching the cartridge holder 120 to the dosing apparatus 110 for assembly, the two leftmost pins 114 are not pushed upwards sufficiently by the associated key element 122 to ensure that also the attached rods 116 are pushed upwards sufficiently to stay above the associated return features 124 of the cartridge holder 120. Instead, the two leftmost rods 116 overlap with the two leftmost return features 124, extending downwards on the right hand side of these return features 124.

As a result, the two leftmost rods 116 act as blocking elements, since they prevent that the cartridge holder 120 can be screwed into the dosing and delivery apparatus 110, as indicated by arrows 160.

It is to be understood that instead of three pins 114 and associated key elements 122, any other number could be used as well.

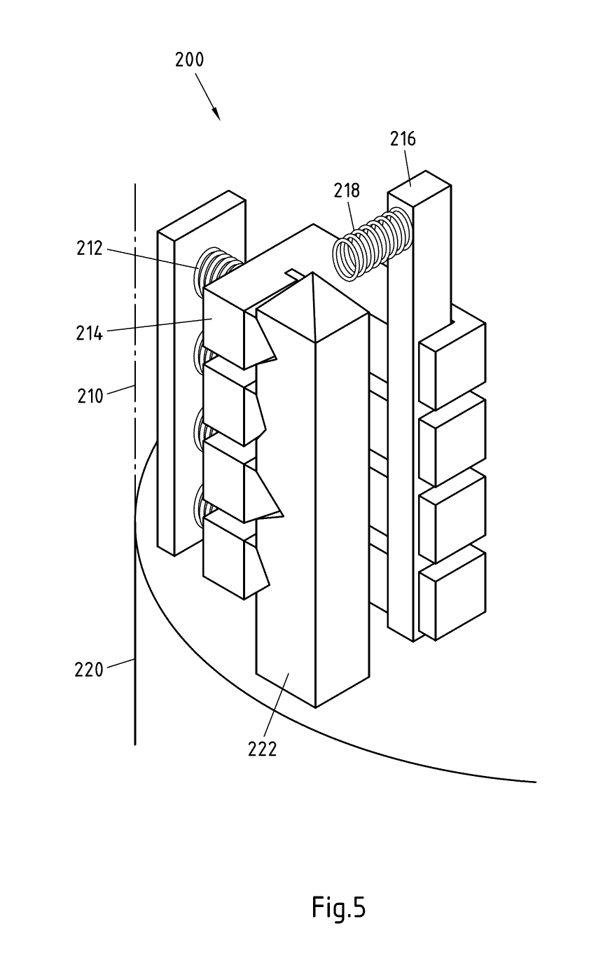

FIGS. 5 to 7 illustrate an assembly of a drug delivery device 200 in accordance with an exemplary second embodiment of the invention.

FIG. 5 is a schematic diagram, which presents selected components of the drug delivery device 200. The drug delivery device 200 could be again an injection pen, an infusion pump, an inhaler, and/or the like.

The presented components comprise a dosing and delivery apparatus 210 depicted in the upper part of the diagram and a cartridge holder 220 depicted in the lower part of the diagram. The dosing and delivery apparatus 210 and the cartridge holder 220 including a cartridge have to be assembled to obtain a functional drug delivery device 200. The components can only be assembled, in case a key part of the cartridge holder 220 is matched to a key validation part of the dosing and delivery apparatus 210.

The dosing and delivery apparatus 210 comprises again conventional components of a dosing apparatus not shown. The dosing and delivery apparatus 210 comprises in addition a number of spring elements 212, for instance three, arranged one above the other and extending horizontally. They can be fixed for instance to the wall of the dosing and delivery apparatus 210. A pin element 214 is attached to a free end of each of the spring elements 212. The combination of spring elements 212 and pin elements 214 form the key validation part of the dosing and delivery apparatus 210. The dosing and delivery apparatus 210 further comprises a blocking element 216 and a spring element 218 acting on the blocking element 216. The spring element 218 could equally be attached to the wall of the dosing and delivery apparatus 210, but at an angular distance to the spring elements 212. The combination of blocking element 216 and spring element 218 forms a safety part of the dosing and delivery apparatus 210. As can be seen, the spring element 218 is arranged above the elements of the key validation part, while the blocking element 216 is a beam that extends downwards, starting from the height of the spring element 218, over the entire height of the key validation part.

The cartridge holder 220 comprises a key part 222 interacting with the key validation part of the dosing and delivery apparatus 210 when the upper part of the cartridge holder 220 is brought together with the lower part of the dosing apparatus 210 such that the key part 222 enters the dosing apparatus 210.

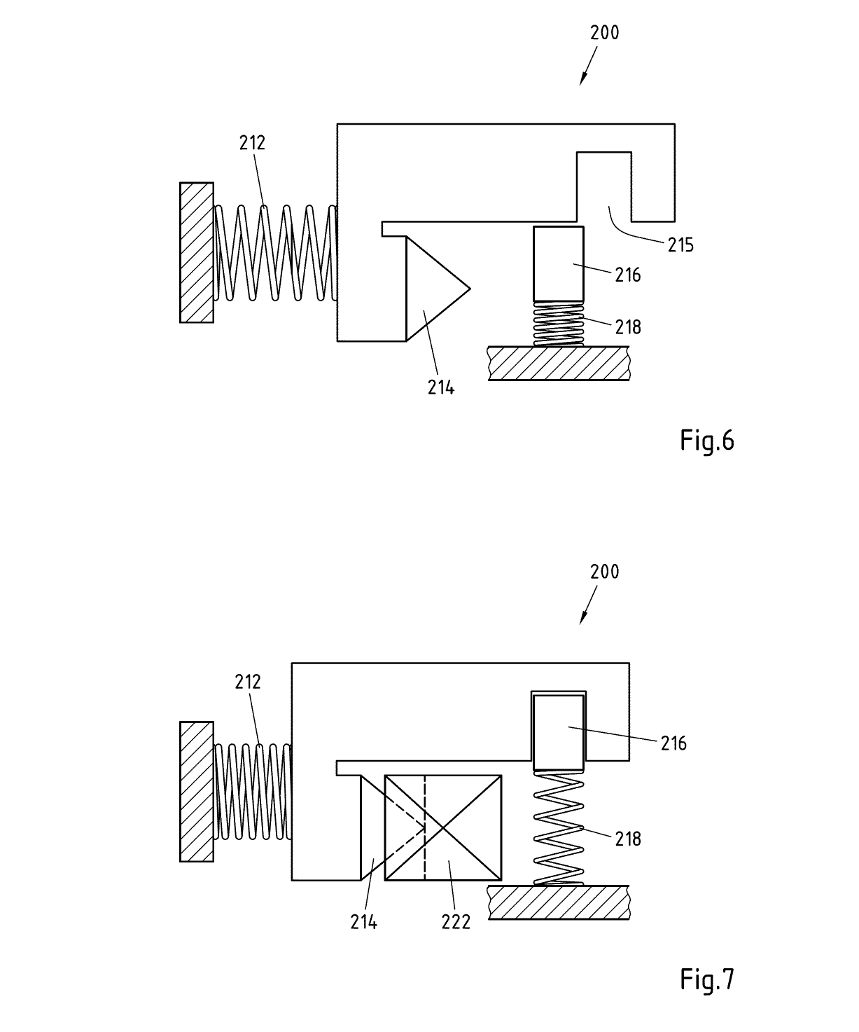

The interaction between the key part 222 and the key validation part 212, 214 will now be described in more detail with reference to FIGS. 6 and 7.

FIG. 6 is a schematic diagram of a top view on one pair of a spring element 212 and a pin element 214 of the key validation part of the dosing and delivery apparatus 210 and on the blocking element 222 and the spring element 218 of the dosing and delivery apparatus 210.

Each pin element 214 has a short, pointed part for interacting with a key part and in parallel a longer rectangular part including a recess 215 for interacting with the blocking element 216. There is a short connection between the pointed part and the rectangular part where the pin element 214 is connected to the spring element 212.

While no key part has been entered into the dosing and delivery apparatus 210, the spring element 212 is decompressed. As a result, the blocking element 216 is pushed back by the rectangular part of the pin element 214 so that the spring element 218 is compressed.

If a key part 222 of a cartridge holder 220 is entered, as shown in FIG. 7, each pin 214 will be pushed towards the housing of the dosing and delivery apparatus 210 so that the associated spring element 212 is compressed. As a result, the recess 215 of each pin elements 214 is moved in direction of the housing of the dosing and delivery apparatus 210 as well.

If the key part 222 of cartridge holder 220 is matched to the key validation part of dosing apparatus 210, each of the pin elements 214 is pushed exactly by an amount that ensures that the associated recess 215 aligns with the blocking element 216. When the recesses of all pin elements 214 are aligned with the blocking element 216 and thus to each other, the compressed spring element 218 pushes the blocking element 216 into the series of recesses 215. As a result, the blocking element 216 clears the passage for the key part 222, so that a user is able to screw the cartridge holder 220 into the dosing and delivery apparatus 210.

It is to be understood that some mechanism may be provided to retract the blocking element 216 from the recesses 215 again, when the cartridge holder 220 and thus the key part 222 are removed again from the dosing and delivery apparatus 210. For instance, the left hand side of the blocking element 216 in the top view of FIGS. 6 and 7 could be slanted such that the upper side of the blocking element 216 is smaller than the lower side of the blocking element 216, which is attached to the spring 218. Alternatively or in addition, the right wall of the recess 215 in the top view of FIGS. 6 and 7 could be slanted in a complementary manner. When retracting the key part 222, the force of the spring elements 212 pushing the pin elements 214 forward could then push the blocking element 216 out of the recess 215, against the force applied by the spring element 218.

If the key part 222 of the cartridge holder 220 is not matched to the key validation part of the dosing and delivery apparatus 210, at least one of the pin elements 214 will be pushed by the key part 222 by an amount that does not ensure that the associated recess 215 aligns with the blocking element 216. In this case, the compressed spring element 218 is not able to move the blocking element 216. As a result, the blocking element 216 continues blocking the passage of the key part 22 and prevents thereby that the cartridge holder 220 is screwed in to the dosing and delivery apparatus 210.

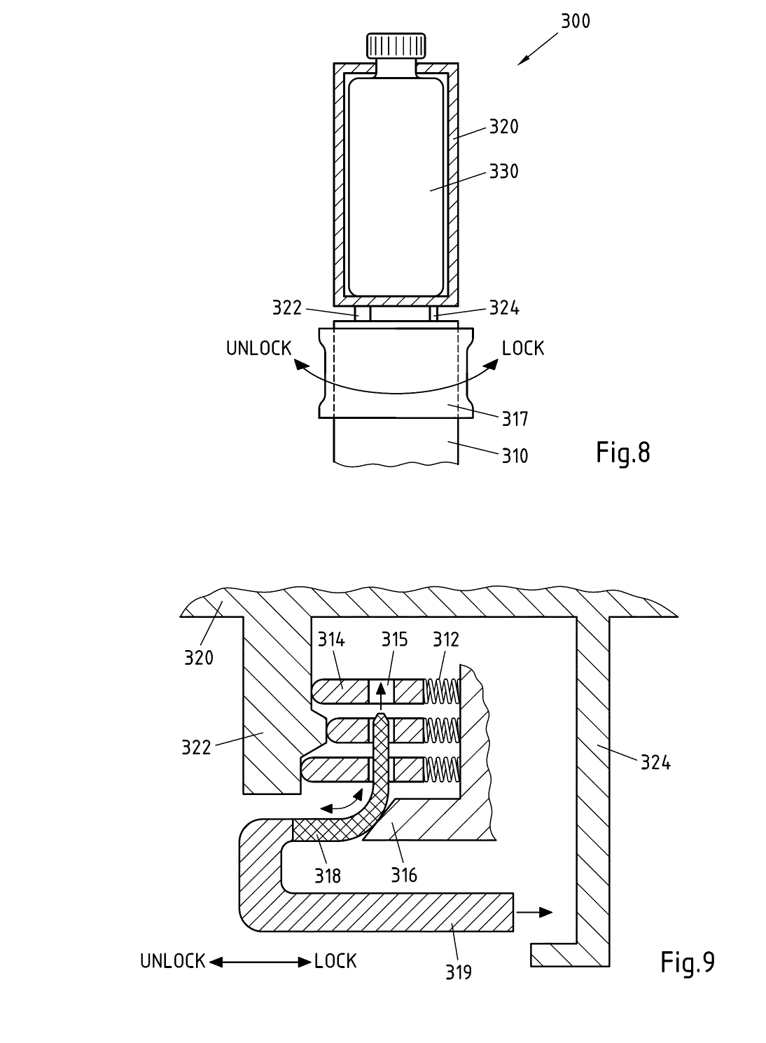

FIGS. 8 to 10 illustrate an assembly of a drug delivery device 300 in accordance with an exemplary third embodiment of the invention.

FIG. 8 is a schematic diagram of a drug delivery device 300 that is about to be assembled. The drug delivery device 300 comprises a delivery apparatus 310 depicted in the lower part of the diagram, and a cartridge holder 320 including a cartridge 330 depicted in the upper part of the diagram.

The delivery apparatus 310 may comprise again conventional components of a delivery apparatus not shown. The delivery apparatus 310 comprises in addition a key validation part. The delivery apparatus 310 further comprises a lock/unlock ring 317. The lock/unlock ring 317 surrounds the upper end of the dosing apparatus 310 and is fixed to the housing of the dosing apparatus 310 such that it can be turned while staying at the same height. The lock/unlock ring 317 comprises at its inner surface a safety part and a retainer part, both extending into the actual delivery apparatus 310, for example through an opening in the housing of the delivery apparatus 310.

The cartridge holder 320 comprises a key part 322 that extends into the delivery apparatus 310 when the drug delivery device 300 is assembled. In addition, the cartridge holder 320 comprises a retainer counterpart 324 that equally extends into the delivery apparatus 310 when the drug delivery device 300 is assembled. A small portion of key part 322 and retainer counterpart 324 are still visible in FIG. 8, since cartridge holder 320 and dosing apparatus 310 have not yet been pushed together completely as required for a complete assembly.

FIG. 9 is a schematic diagram illustrating the interaction between key part, key validation part, safety part, retainer part and retainer counterpart of the drug delivery device 300 of FIG. 8.

The key part 322 and the retainer counterpart 324 are attached to the cartridge holder 320 and extend into the delivery apparatus 310, as mentioned before. The right hand side of the key part 322 has a profiled surface structure, which is specific to the cartridge holder 320 and thus indirectly to the cartridge 330 and the contained medicament. The retainer counterpart 324 can simply be a vertical rod, which is bended at its lower end towards the left hand side.

The key validation part comprises a number of spring elements 312, for instance three, that are attached one above the other to the housing of the delivery apparatus 310 and extend vertically to the left hand side. Moreover, the key validation part comprises pin elements 314 attached to the free end of a respective spring element 312 and extending still further to the left hand side. Each of the pin elements 314 has a recess 315 passing through the pin element 314 from top to bottom, for instance in the form of a circular hole. The pin elements 314 may have different lengths and the recess 315 of each pin element 314 may be provided at a different location. Finally, the key validation part may have a guiding element 316, which is equally fixed to the housing of the delivery apparatus 310.

The safety part is attached to the inner surface of the lock/unlock ring 317 and comprises a flexible arm 318 as a blocking element as well as a rigid arm 319 as a retainer part.

When cartridge holder 320 and delivery apparatus 310 are brought together, the key part 322 pushes the pin elements 314 in direction of the housing of the delivery apparatus 310, compressing the spring elements 312. The amount of movement of each pin elements 314 is determined by the profile of the key part 322 at the height of the pin element 314 when cartridge holder 320 and delivery apparatus 310 have been brought together completely.

In case the key part 322 is matched to the key validation part, the recesses 315 of the pin elements 314 align to form a passageway, as shown in FIG. 9. As a result, the flexible arm 318 can be pushed through the formed passageway, guided by the guiding element 316, by turning the lock/unlock ring 317 counterclockwise. As the flexible arm 318 is pushed through the passageway, the rigid arm 319 will be moved by the same amount in direction of the retainer counterpart 324 until it overlaps with the bend portion of the retainer counterpart 324. As a result, the cartridge container 320 is securely fixed to the delivery apparatus 310. The fixing can be lifted again simply by turning the lock/unlock ring 317 in a clockwise rotation.

In case the key part 322 is not matched to the key validation part, in contrast, the recesses 315 of the pin elements 314 will not align and no passageway is formed. As a result, the lock/unlock ring 317 cannot be turned counterclockwise as far as with a matching key part 322, since the flexible arm 318 will be stopped at the latest by the uppermost pin element 314. Consequently, the rigid arm 319 cannot be moved sufficiently to overlap with the bend portion of the retainer counterpart 324 either, and the cartridge container 320 cannot be securely fixed to the delivery apparatus 310.

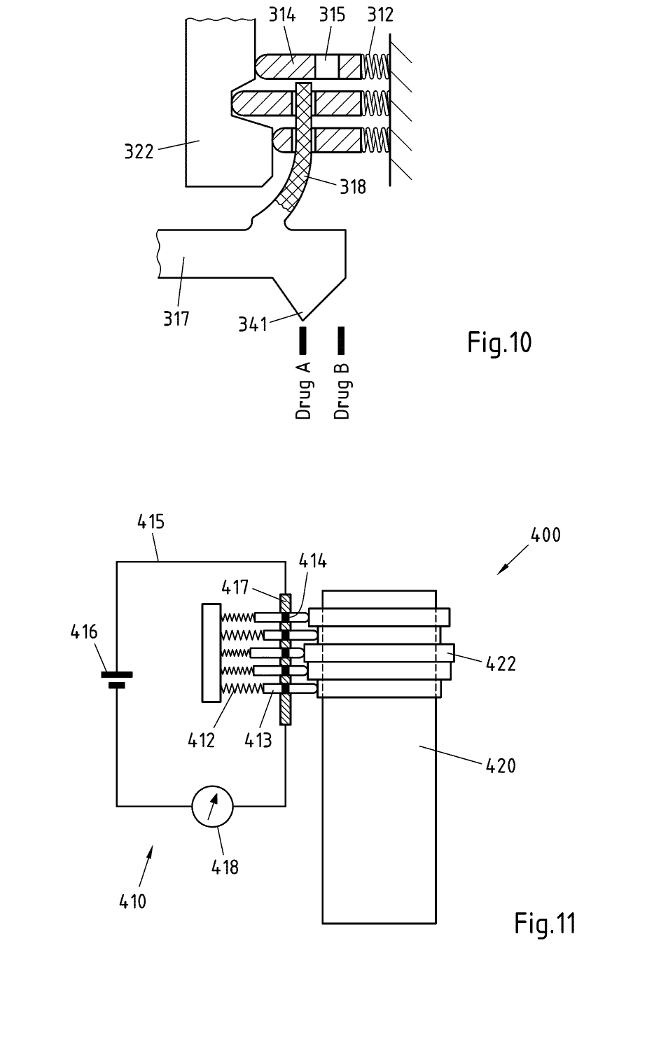

FIG. 10 is a schematic diagram presenting a variation of or a supplement to the third embodiment presented with reference to FIGS. 8 and 9. Like elements are denoted with the same reference signs.

This variation includes as well a cartridge holder with a key part 322, and a delivery apparatus with a key validation part including spring elements 312 and pin elements 314 with a respective recess 315, and with a safety part including a flexible arm 318 fixed to a lock/unlock ring 317 of the delivery apparatus 310. The other elements described for the embodiment of FIGS. 8 and 9 may or may not be included as well.

In this variation, the lock/unlock ring 317 comprises in addition an indicator 341 that is visible to a user. The indicator 341 could be simply a triangular form attached to the bottom of the lock/unlock ring 317, with one corner pointing downwards similarly as an arrow. Moreover, the delivery assembly comprises a marking on its housing beneath the lock/unlock ring 317. The marking could include for example the name of a first medicament “Drug A” and the name of a second medicament “Drug B”.

Now, a key part 322 of a first type of cartridge holder may align the recesses 315 of the pin elements 314 such that the flexible arm 318 may be pushed through all of the pin elements 314, similarly as shown in FIG. 9. In this case, the indicator 341 comes to stop above the marking “Drug B”. A key part 322 of a second type of cartridge holder, in contrast, may align the recesses 315 of the pin elements 314 such that the flexible arm 318 may be pushed through all but the highest one of the pin elements 314, as shown in FIG. 10. That is, the lock/unlock ring 317 cannot be turned quite as far as with the key part 322 of the first type of cartridge holder. In this case, the indicator 341 comes to stop above the marking “Drug A”. If a retainer counterpart is provided in this variation as in FIG. 9, the bended part could be sufficiently long so that a secure fix can be achieved with both types of key parts.

FIG. 11 illustrates an assembly of a drug delivery device 400 in accordance with an exemplary fourth embodiment of the invention. FIG. 11 is a schematic diagram, which presents selected components of the drug delivery device 400. The drug delivery device 400 could be for instance an injection pen.

The presented components belong to a dosing and delivery apparatus 410 and a cartridge 420, which have to be assembled to obtain a functional drug delivery device 400.

The dosing and delivery apparatus 410 comprises again conventional components of a delivery apparatus not shown. The dosing and delivery apparatus 410 comprises in addition a key validation part. The key validation part includes five springs 412 that are fixed within the dosing and delivery apparatus 410. The key validation part includes in addition five pins 413, each being attached to the free ends of one of the five springs 412. Each of the pins 413 comprises a conductive element 414 somewhere in the middle. Springs 412 and pins 413 extend horizontally in the depicted arrangement. The dosing and delivery apparatus 410 comprises in addition a safety part. The safety part comprises an electrical circuit 415. The electrical circuit 415 includes a power source 416, a conductive element 417 and a sensor 418. The power source 416 could be a battery, for instance the same battery that is used for driving a motor of the dosing and delivery apparatus 410. The conductive element 417 is made of a conductive material that is interrupted at five positions by a non-conductive material or a less conductive material. The conductive element 417 is arranged to extend vertically to the horizontally arranged pins 413. Each of the areas made of non-conductive material is in close contact with one of the pins 413, the breadth of the pins 413 overlapping the areas made of non-conductive material.

The cartridge 420 may be used by itself or it may be included in a cartridge holder, not shown. The upper end of the cartridge 420 is surrounded by five fixed key rings 422. The inner diameter of each of the key rings 422 is basically the same as the outer diameter of the cartridge cylinder. The outer diameters of the key rings 422 may differ from each other to enable a large number of different codings for different types of cartridges. Using key rings 422 has the effect that the cartridge 420 could be inserted into a dosing and delivery apparatus 410 with any rotational angle. It is to be understood, though, that the cartridge could equally be provided for example with pins attached to one side of the cartridge only, in particular if guidance elements are provided which ensure that the cartridge 420 can be inserted into the dosing and delivery apparatus 410 with a single rotational orientation only.

When the cartridge 420 is inserted into the dosing and delivery apparatus 410, each key ring 422 pushes back one of the pins 413, compressing the associated spring 412. If the outer diameters of the key rings 422 are matched to the pins 413, the conductive areas 414 of the five pins 413 align exactly on top of the five non-conductive areas of the conductive element 417. As a result, the electrical circuit 415 is closed. The pins 413 thus act as a kind of a switch for the electrical circuit 415. The sensor 418 will sense the closing of the electrical circuit 415 and send a signal indicating that a correct cartridge has been inserted to a control part of the dosing and delivery device 410, not shown. Only upon receipt of such a signal, the control part will enable an activation of the drug delivery device 400, for example an activation of a motor, a dose selection function, and/or any other functional part used to deliver the drug or medicament of the drug delivery device 400.

Alternatively or in addition, the safety part could comprise an indicator. Such an indicator could be for instance a light-emitting diode and replace the sensor 418 in electrical circuit 415. A closed circuit 415 could then power up the light emitting diode to indicate to a user that a correct cartridge has been inserted. The indicator could also be provided externally to the electric circuit 15 and be caused by sensor 418 to inform a user whenever a correct cartridge has been inserted.

In another variation, the dosing and delivery apparatus 410 could support a plurality of cartridge types. In this case, at least one of the pins 413 could be provided with at least two conductive parts. If these conductive parts have furthermore different electric properties, such as different resistances, the sensor 418 could detect which type of cartridge has been inserted and indicate so by activating one of different light emitting diodes associated to different drugs or by providing an indication on a display of the dosing apparatus 410. In another variation, the closed electrical circuit 415 could power up a normally closed locking pin to enable a complete assembly of the drug delivery device in the case of matched key rings. In yet another variation, at least a part of the electrical circuit 415 could be integrated in a cartridge holder. It is further to be understood that any other number of key rings 422 and associated pins 413 could be used.

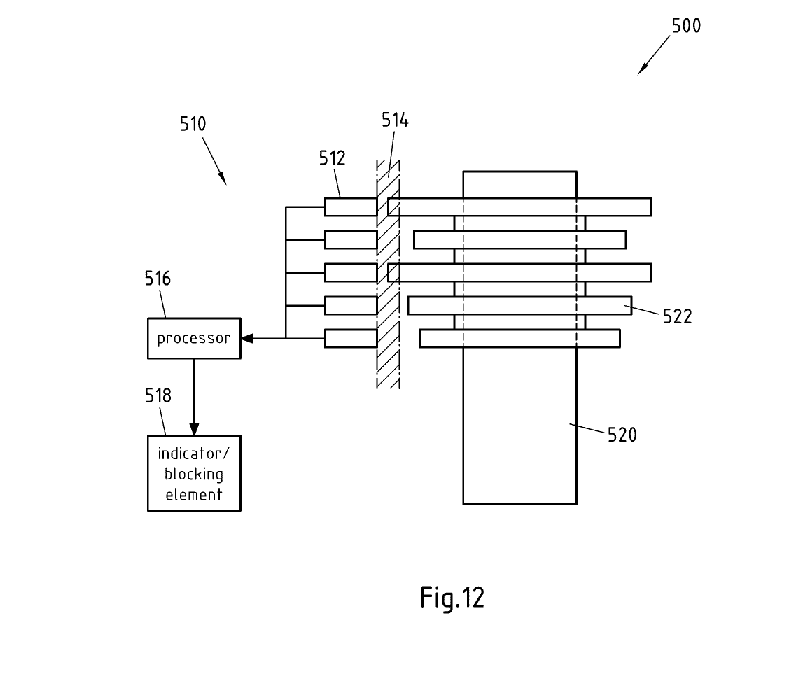

FIG. 12 illustrates an assembly of a drug delivery device 500 in accordance with an exemplary fifth embodiment of the invention. The arrangement is similar as in the fourth embodiment described with reference to FIG. 11.

A cartridge 520 may be provided again with a number of key rings 524, for instance five, or similar coding means.

The dosing and delivery apparatus 510 comprises in this case a key validation part including a series of proximity sensors or proximity switches 512 and a processor 516, though. Each proximity switch 512 is able to detect the presence of an element in a sensing distance 514. The processor 516 is configured to receive and evaluate the signals provided by the proximity switch 512. The dosing and delivery apparatus 510 comprises in addition a safety part 518 linked to the processor 516. The safety part 518 may comprise indicator means and/or blocking means.

If the cartridge 520 is inserted into the dosing and delivery apparatus 510, the proximity switches 512 will emit a signal ‘10100’, where a ‘1’ stands for ‘on’ and a ‘0’ for ‘off’ of a respective one of the five switches 512, since the first and third ones of the key rings 522, counted from the top, extends into the sensing distance 514, while the second, fourth and fifth ones of the key rings 522 do not extend into the sensing distance 514. In an alternative embodiment, the proximity switches 512 are sensors, each sensor emitting a signal indicative of a sensed distance 514 of a key ring 522. The processor 516 may evaluate whether each ring 522 is within or at a predefined distance, or whether each ring is within a predefined distance range.

The processor 516 may determine based on these signals whether the inserted cartridge 520 is of a supported type, and possibly in addition determine the type of the inserted cartridge 520 in case a plurality of different cartridge types using different key rings 522 is supported. The result of the evaluation by the processor 516 may be used for causing the safety part 518 to indicate that a correct cartridge is being used, to indicate which type of cartridge is being used, to block or enable a further assembly of the drug delivery device 500, and/or to prevent or enable an operation of the drug delivery device 500, similarly as described for the fourth embodiment.

It is to be understood that the presented embodiments can be varied in many ways within the scope of the appended claims.

In particular, the arrangement of key part, key validation part and safety part could be distributed differently to components of a drug delivery device, for example to cartridge, cartridge holder and delivery apparatus. Furthermore, any mentioned spring element could be replaced by some other type of biasing element. Moreover, the features of any presented embodiment can be combined with the features of any other presented embodiment.

1. A component for a drug delivery device, the component comprising:
a key validation part configured to interact with a key part of another component of the drug delivery device during an assembly of the component with the other component, wherein the key validation part comprises at least one pin attached to at least one biasing element, the at least one pin being configured to be moved, against a force applied by the at least one biasing element, by the key part of the other component; and
a safety part configured to have a different impact if the key part is matched to the key validation part compared to if the key part is not matched to the key validation part, wherein the safety part comprises a blocking element, and wherein the blocking element is configured to prevent, as one impact, further assembly of the drug delivery device in case the at least one pin is not moved sufficiently by the key part of the other component.

2. The component according to claim 1, wherein the blocking element is connected to the at least one pin and/or to the at least one biasing element.

3. The component according to claim 1, wherein the at least one pin comprises at least one recess, the at least one pin being configured to be moved by the key part matching the key validation part to a release position at which the blocking element is enabled to enter the at least one recess to enable, as the different impact, a full assembly of the component with the other component.

4. The component according to claim 3, wherein the safety part further comprises at least one biasing element configured to push the blocking element into the at least one recess if the at least one pin has been moved by the key part of the other component into the release position.

5. The component according to claim 3, wherein the blocking element comprises a lock/unlock ring comprising a flexible arm, the flexible arm being configured to move into the at least one recess when turning the lock/unlock ring, if the at least one pin has been moved by the key part of the other component into the release position.

6. The component according to claim 1, wherein the safety part comprises an electrical circuit including a sensor, wherein the at least one pin is made at least partially of a conductive material, wherein the key validation part and the electrical circuit are configured such that the resistance of a part of the electrical circuit changes if the at least one pin is moved by a key part matching the key validation part, and wherein the sensor is configured to detect a change in resistance.

7. The component according to claim 1, wherein the at least at least one blocking element is configured to enable, as the different impact, a full assembly of the component with the other component if the key part of the other component is matched to the key validation part.

8. The component according to claim 1, wherein the safety part comprises an indicator configured to indicate, as the different impact, exclusively if the key part of the other component is matched to the key validation part, that the other component is one of at least one other component allowed to be assembled with the component.

9. The component according to claim 1, wherein the safety part comprises an indicator configured to identify the other component to a user, as the different impact, exclusively if the key part of the other component is matched to the key validation part.

10. The component according to claim 1, wherein the component comprises at least one of
a drug delivery apparatus;
a drug dosing and delivery apparatus;
a cartridge configured to comprise a drug; and
a cartridge holder.

11. A drug delivery device comprising the component according to claim 1 and the other component according to claim 1.

12. A component for a drug delivery device, the component comprising:
a key validation part configured to interact with a key part of another component of the drug delivery device during an assembly of the component with the other component, wherein the key validation part comprises at least one proximity sensor and a processor, the at least one proximity sensor configured to detect whether the key part of another component comes to lie within or at a predetermined distance to each of the at least one proximity sensor during an assembly of the component with the other component, and wherein the processor is configured to determine whether the key part of the other component matches the key validation part by evaluating the output of the at least one proximity sensor; and
a safety part configured to have a different impact if the key part is matched to the key validation part compared to if the key part is not matched to the key validation part.

13. The component according to claim 12, wherein the safety part is configured to prevent, as one impact, an operation of the drug delivery device if the key part of the other component is not matched to the key validation part and to enable, as the different impact, an operation of the assembled device if the key part of the other component is matched to the key validation part.
